(19) United States
(12) Patent Application Publication
(10) Pub. No.: US 2008/0261916 A1
(43) Pub. Date: Oct. 23, 2008

(54) Synergistic Prebiotic Compositions

(76) Inventors: Csaba Jozsef Jaszberenyi, Budapest (HU); Tamas Janos Szakacs, Debrecen (HU)

Correspondence Address: HAHN & VOIGHT PLLC, 1012 14TH STREET, NW, SUITE 620, WASHINGTON, DC, 20005, US

(21) Appl. No.: 11/917,236

(22) PCT Filed: Jun. 12, 2006
(86) PCT No.: PCT/HU06/00051

§ 371 Date: Mar. 21, 2008

(30) Foreign Application Priority Data
Jun. 13, 2005 (HU) P05 00582

(51) Int. Cl. A61K 31/716; A61K 31/715; A61P 35/00; A61P 15/00; A61K 31/718

(52) U.S. Cl. 514/58; 514/60; 514/54

(57) ABSTRACT

The invention relates to synergistic compositions comprising prebiotic components selected from fructose polymers GFₙ and Fₘ, either containing a glucose (G) end-group, or without a glucose end-group, and one or more component of a group of prebiotics consisting of modified or unmodified starch and partial hydrolysates thereof, partially hydrolysed inulin, natural oligofractoses, fructo-oligosaccharides (FOS), lactulose, galactomannan and suitable partial hydrolysates thereof, indigestible polydextrose, acemannan, various gums, indigestible dextrin and partial hydrolysates thereof, trans-galacto-oligosaccharides (GOS), xylo-oligosaccharides (XOS), beta-glucan and partial hydrolysates thereof, together if desired with phytosterol/phytostanol components and their suitable esters, and if desired other plant extracts, mineral components, vitamins and additives.

The present invention relates to synergistic prebiotic compositions in which fructose polymers of GFₙ or Fₘ structures, either containing a glucose (G) end-group and one or more prebiotic components from a group of prebiotics consisting of modified or unmodified starch and suitable partial hydrolysates thereof, partially hydrolysed inulin, natural oligofructoses, fructo-oligosaccharides (FOS), lactulose, galactomannan and suitable hydrolysates thereof, indigestible polydextrose, indigestible dextrin and partial hydrolysates thereof, trans-galacto-oligosaccharides (GOS), xylo-oligosaccharides (XOS), acemannan, lentinan or beta-glucan and partial hydrolysates thereof, polysaccharides P and K (PSP, PSK), tagatose and if desired phytosterols and lecithins are used, optionally with other plant extracts or dried plant powders, mineral components vitamins, amino acids and other additives.

Prebiotics are in most cases oligo- and/or polysaccharides that are not digested in the stomach and small intestine and reach the colon more or less intact (Roberfroid MB. Prebiotics: preferential substrates for specific germs? American Journal of Clinical Nutrition, 2001: 73(2) 406S-409S). However, colonic bacteria are able to use these compounds (Macfarlane G T, Gibson GR. Metabolic activities of the normal colonic flora. In: Gibson SAW, ed. Human health—the contribution of microorganisms. London: Springer-Verlag, 1994: 17-52). The advantageous prebiotic components are capable to increase the amount of probiotic microorganisms in the colonic microflora (Collins M D, Gibson G R. Probiotics, prebiotics, and synbiotics: approaches for modulating the microbial ecology of the gut, American Journal of Clinical Nutrition, 1999: 69(5), 1052S-1057S). Together with probiotics-related or independent effects said compositions exert a complex physiological influence in the host (Fedorak R N, Madsen K L. Probiotics and prebiotics in gastrointestinal disorders. Current Opinion in Gastroenterology, 2004: 20(2): 146-155). These include the positive change in lipid and cholesterol levels, limiting the occurrence and amount of dangerous or disadvantegous resident bacteria (these include the dangerous Helicobacter pylori or clostrdia) or invading colonic bacteria (Alm L. The effect of Lactobacillus acidophilus administration upon survival of Salmonella in randomly selected human carriers. Prog Food Nutr Sci 1983: 7:13-7; Gibson G R, Wang X. Regulatory effects of bifidobacteria on other colonic bacteria. J Appl Bacteriol 1994; 77:412-20). The said compositions can thereby reduce the risk of the leading causes of death, (Functional Foods, G. R. Gibson, C. M. Williams, eds., 389. pp., Woodhead Publishing Ltd, Abington Hall, Abington, Cambridge, England, 2000/2002) heart and circulatory diseases and colorectal (Reddy B S, Hamid R, Rao C V. Effect of dietary oligofructose and inulin on colonic preneoplastic aberrant crypt foci inhibition. 1997: Carcinogenesis 18:1371-1374) or other cancer (Van Loo J, Clune Y, Bennett M, Collins J K. The SYNCAN project: goals, set-up, first results and settings of the human dietary intervention study. 2005: Brit. J. Nutr. 93(S1), 91-98). Interestingly, the anticancer properties of prebiotics are not limited to colonic events (Taper H S, Roberfroid M. Influence of inulin and oligofructose on breast cancer and tumor growth. J. Nutr. 1999: 129:1488 S-1491S.) An important part of human nutrition is the consumption of suitable amount of soluble and insoluble fiber. It is known from the literature that it is possible to influence and change the colonic microflora thereby improving the health of the host. The prebiotic-probiotic-synbiotic concept is a clear demostration of these effects (Bengmark S. Pre-, pro- and synbiotics. Current Opinion in Clinical Nutrition and Metabolic Care 2001: 4(6):571-579; Bengmark S. Gut microbial ecology in critical illness: is there a role for pre-, pro-, and synbiotics. Current Opinion in Critical Care, 2002: 8: 2). The gastro-intestinal system of a fetus is sterile. The colonization starts during and after birth and the formation of the complex colonic microflora proceeds for years. In fact, the colonic microflora is a changeable biodynamic ecosystem. By the age of two years, the colon of a child is colonized with a hundred or a couple of hundreds strains of bacteria. This develops further and the colonic microflora then remains a close-knit commensal bacterial ecosystem until the age of about 60 years. After that this systems gets somewhat loose, allowing new (unwanted) members to get attached to the system. This contributes or may contribute to the decline of health of the elderly people. The microbes in the colon (colonized and planktonic) are capable to exert the following positive effects:
Suppressing the dangerous colonic microorganisms (invading or colonized) and thereby the production of some of their potentially carcinogenic metabolism products and certain unwanted enzymes).
Prevention of the colon from attack of dangerous exogenous microorganisms.
Increase of the ratio of the advantageous probiotic bacteria both in the small intestine and also in the colon.
Improving the developing colonic microflora of the newborn.
Prevention of the outbreak and reducing the severity of the symptoms of diarrhea caused by rotaviruses and other viruses and bacteria.
Reducing the symptoms of chronic intestinal inflammations (Crohn-disease, IBS, colitis).
Production of short-chain fatty acids (SCF).
Production of lactic acid.
Influence on bile-acid transport and biochemical transformations thereof.
Partial inhibition of hepatic cholesterol biosynthesis.
Modulation of insulin production and activity.
Providing extra food for the colonocytes.
Regulating the production of immunoglobulins and secretory IgA.
Suppression of the formation of aberrant crypt foci and further steps of colorectal carcinogenesis.
Suppression of cancer-causing potential of external chemical carcinogenes.
Suppression of clostridial formation of potentially carcinogenic secondary bile acids.
Suppression of allergies (including atopic dermatitis).
Reducing the symptoms of lactose intolerance and other food allergies.
Reducing the pH of the colon, thereby making the conditions less suitable for certain pathogens.
Increasing bowel motility.
Increasing calcium uptake.

These processes, through various biochemical and physiological pathways, exert a generally advantageous physiological action for the host (in this case the human body). It is noteworthy, however, that said positive physiological effects can also be useful for animals. Therefore, the compositions can also be utilized in fodder and feed additives (Abe F, Ishibashi N, Shimamura S. Effect of administration of bifidobacteria and lactic acid bacteria to newborn calves and piglets. J. Dairy Sci. 1995: 78:2838-2846.). In addition to the bioactivity discussed above, said compositions are capable to reduce symptoms of allergy (Noverr M C, Huffnagle GB. Does the microbiota regulate immune responses outside the gut? Trends in Microbiology 2004: 12:562-568). Said compositions are able to positively modulate the immune system (Gut Flora, Nutrition and Immunity, Fuller R., Perdigón G., Eds., Blackwell Publishing, 2003).

It is well documented in the literature that phytosterols as well as their hydrogenated counterparts, phytostanols and their esters are capable to reduce the total cholesterol and low density lipoprotein cholesterol (LDL) levels in the human blood. Consumption of plant sterols, however, is not a new phenomenon. In fact, it precedes that of the human development. Our close relative primates continue to consume a much higher amount of phytosterols daily than the modern man. The typically 100 to 300 mg/day plant sterol consumption of humans today is much less than the amount required to achieve a meaningful change in blood lipid parameters.

The effect is complex. It is achieved by the phytosterol inhibition of the absorption of the exogenous (food) cholesterol and also by the inhibition of the reabsorption of the cholesterol transported by the endogenous enterohepatic circulation. These result in increased cholesterol clearance. The cholesterol and phytosterol transport can be modulated by the application of plant sterols in lecithin micelles (Ostlund R E, Jr., Spilburg C A, Stenson W F. Sitostanol administered in lecithin micelles potentially reduces cholesterol absorption in humans. American Journal of Clinical Nutrition, 1999: 70:826-831). Phytosterols are also capable to prevent the development of benign prostatic hyperplasia (BPH) (Wilt T J, MacDonald R, Ishani A. Beta-sitosterol for the treatment of benign prostatic hyperplasia: a systematic review. BJU Int. 1999; 83:976-983). The successful and efficient application of phytosterol esters in bakery products for the reduction of plasma LDL-cholesterol has also been documented (Quilez J, Rafecas M, Brufau G, Garcia-Lorda P, Megias I, Bulló M, Ruiz J A, Salas-Salvado J, J. Nutr. 2003: 133:3103-3109 and references there cited).

We have found that a proper combination of prebiotics may exert a synergistic effect. The compositions comprising the combination according to the present invention may be used as medicaments, cosmetics, food and fodder additives, dietary supplements, as well as prebiotic and symbiotic food and fodder.

The present invention relates to synergistic prebiotic compositions comprising prebiotic components selected from fructose polymers GFₙ and Fₘ, either containing a glucose (G) end-group, or without this glucose end-group and one or more component of a group of prebiotics consisting of modified or unmodified starch and partial hydrolysates thereof, partially hydrolysed inulin, natural oligofructoses, fructo-oligosaccharides (FOS), lactulose, galactomannan and suitable partial hydrolysates thereof, indigestible polydextrose, acemannan, various gums, indigestible dextrin and partial hydrolysates thereof, trans-galacto-oligosaccharides (GOS), xylo-oligosaccharides (XOS), beta-glucan and partial hydrolysates thereof, together if desired with phytosterol/phytostanol components and their suitable esters, and if desired other plant extracts, mineral components, vitamins and additives.

Preferably, the compositions according to the present invention comprise prebiotic components selected from fructose polymers GFₙ and Fₘ, either containing a glucose (G) end-group, or without this glucose end-group and one or more component of a group of prebiotics consisting of modified or unmodified starch and partial hydrolysates thereof, partially hydrolysed inulin, natural oligofructoses, fructo-oligosaccharides (FOS), lactulose, galactomannan and suitable partial hydrolysates thereof, indigestible polydextrose, acemannan, various gums, indigestible dextrin and partial hydrolysates thereof, trans-galacto-oligosaccharides (GOS), xylo-oligosaccharides (XOS), beta-glucan and partial hydrolysates thereof, together if desired with phytosterol/phytostanol components and their suitable esters, and if desired other plant extracts, mineral components, vitamins and additives. The fructose polymers of GFₙ or Fₘ structures (G=glucose; F=fructose; n>2; m>2) are linear fructose polymers having either a glucose (G) and -group, or being without this glucose and -group. Oligofructoses are consisted of 3 to 10 carbohydrate units. Above that, chicory inulin contains 10 to 60 carbohydrate units, typically with 27 carbohydrates (fructoses with our without one glucose end-group and a fructose chain). Other plants may produce different fructans. These fructans are capable to increase the number of colonized and planktonic bacteria in the large intestine. This results in a change that those bacteria that are less advantageous or may turn dangerous are suppressed by the higher probiotic colony of bacteria. Depending on the chain length of these fructans or other prebiotics, they can be fermented by probiotic bacteria at different positions in the colon. We have found that the longer inulins are capable to rich the distal colon and sigmoid colon and exert their anticancer actions in the positions where typically most of the cancerous problems occur. The occurrence of these cancers can be the result of various types of carcinogenesis. It has been demonstrated in the literature that directly induced chemical carcinogenesis can be greatly reduced by probiotic bacteria. The prebiotic compositions of our invention can corroborate this effect by considerably increasing the number of Bifidocateria and other beneficial probiotic strains. The local chemical carcinogenesis can also be the result of the formation of secondary bile acids. These secondary bile acids are often formed upon the action of enzymes produced by resident Clostridia. By probiotic suppression of the number of these bacteria according to the invention, the chance of secondary bile acid formation can also be reduced. This can be demonstrated by measuring the faecal primary/secondary bile acid ratio.

Other prebiotics can be selected from a group of prebiotics consisting of various gums (guar gum, xanthan gum, locust been gum), carob seed flour, oat bran, rice bran, barley, modified or unmodified starch and suitable partial hydrolysates thereof, partially hydrolysed inulin, natural or synthetic/biosynthetic oligofructoses, fructo-oligosaccharides (FOS), lactulose, galactomannan and suitable hydrolysates thereof, indigestible polydextrose, indigestible dextrin and partial hydrolysates thereof, trans-galacto-oligosaccharides (GOS), xylo-oligosaccharides (XOS), acemannan, lentinan or beta-glucan and partial hydrolysates thereof, polysaccharides P and K (PSP, PSK), tagatose, various fungal oligosaccharides and polysaccharides, together with other components.

Further embodiment of the invention is the application of various phytosterols in these compositions. These phytosterols can be found in various plants. Typically the mixture of phytosterols used in foods are of soy or tall oil origin. Corn fiber oil is also very rich in phytosterols and their derivatives. It is well documented in the scientific literature that a considerable reduction (10-20%) of low density lipoprotein (LDL) and total cholesterol (TC) can be achieved by suitable administration of these phytosterol mixtures, their reduced (phytostanol) counterparts and the corresponding phytosterol and phytostanol esters. It has also been demonstrated that the sterols and stanols on the one hand and their esterified counterparts on the other are all suitable for this purpose. The question remains whether these compounds are soluble in the media of applications.

A further embodiment of this invention is the formation of supramolecular compositions. In this process a spontaneous multicomponent supramolecular self-assembly (SMSA) takes place between the components (Jean-Marie Lehn, Perspectives in supramolecular chemistry: From molecular recognition towards self-organisation. Pure and Applied Chemistry 1994: 66:1961-1966). To corroborate this self-assembly, we add lecithins and/or an edible oil or a mixture of edible oils, preferably with omega-3-fatty acid content during the preparation of these compositions. This results in improved solubility profile of the composition compared to the starting components. We have observed that the synergic effect of our compositions is achieved not only by the joint application of various prebiotics. In certain cases the synergy can be the result of the use of prebiotics, probiotics, phytosterols and derivatives, various plant extracts and powders, edible plant oils and their diglyceride and monoglyceride counterparts, lecithins, amino acids and minerals in supramolecular structures. These supramolecular structures positively influence their stability and transport properties. This supramolecular arrangement represents new qualities of the original components and further corroborates their useful bioactivities by modified solubility, transport and stability in these supramolecular assemblies.

A further embodiment of the present invention are the cases when these compositions incorporate further components belonging to the group of vitamins. Due to the special process, these vitamins can be both water soluble and water insoluble. This allows us to employ vitamins that can exert their own action and they can also corroborate the actions of the aforementioned prebiotic components and also those of the phytosterols. A very important contribution of these vitamin mixtures can be that of their antioxidant properties.

A further embodiment of the present invention is the incorporation of physiologically important elements and trace elements that include but are not limited to calcium, magnesium, zinc, phosphorus, selenium, boron, chromium, copper, potassium, iodine, indium and other useful trace elements.

In a further embodiment of the present invention various extracts and plant powders are incorporated into our compositions, depending on the desired properties according to the end use of said compositions. These compositions according to the present invention can be characterized in that in addition to the discussed prebiotics and phytosterols and lecithins the said further plant extracts or powders are one or more of those of Panax ginseng (red, Korean ginseng), Panax ginseng (white, Chinese ginseng), Rhodiola rosea (golden root), Panax quinquefolium (American ginseng), Eleutherococcus senticosus (Siberian ginseng), Cynara scolymus (artichoke), Uncaria tomentosa (Cat's claw), Lepidium meyenii (maca, Peruvian ginseng), Paullinia cupana (guarana), Croton lechleri (Sangre de Grado), Whitania somnifera (ashwagandha, Indian ginseng), Panax japonicus (Japanese ginseng), Panax vietnamensis (Vietnamese ginseng), Panax trifolius, Panax pseudoginseng, Panax notoginseng, Malpighia glabra (acerola), Ylex paraguayiensis (Yerba mate), Astragalus membranaceus (astragalus), Stevia rebaudiana (stevia), Pfaffia paniculata (Brazilian ginseng, suma), Ginkgo biloba, Tabebuia impetiginosa (Pau d'arco), Echinacea purpurea, Peumus boldus (boldo), Gynostemma pentaphyllum (Jiaogulan, also known as Southern Ginseng or Xiancao), Sutherlandia frutescens (African ginseng), Aloe vera (aloe), Cistanche salsa, Cistanche deserticola (and other Cistanche sp.), Codonopsis pilosula (“poor man's ginseng.”), Nopal opuntia (Prickly pear cactus), Citrus sinensis (Citrus aurantium) and other members of the citrus family (lemon, lime, tangerine, grapefruit), Camelia sinensis (tea), Plantago psyllium (psyllium), Amaranth edulis and other amaranth sp. (amaranth), Commiphora mukul (guggul lipid), Serenoa repens, Serenoa serrulata (saw palmetto), Cordyceps sinensis (Cordycaps), Lentinula edodes (Shitake), Ganoderma lucidium (Reishi), Grifola frondosa (maitake), Tremella fuciformis (Silver ear), Poria cocos (Hoelen), Hericium erinaceus (Lion's Mane), Agaricus blazei (Sun mushroom), Phellinus linteus (Mulberry yellow polypore), Trametes versicolor, Coriolus versicolor (Turkey tails), Schizophyllum commune (Split gill), Inonotus obliquus (Cinder conl), oat bran, rice bran, linseed, garlic, Ceratonia siliqua (locust been gum or flour from the seeds of carob tree), Cyanopsis tetragonoloba (guar gum, EU Food additive code E412), Xanthomonas campestris (xanthan gum). These plant extracts and plant powders are capable to potentiate the bioactivity of these compositions based on prebiotics, phytosterols, lecithins, vitamins and minerals. In given cases it also adds other prebiotics to the aforementioned prebiotic mixtures. These can result in more pronounced bioactivities as prebiotics and also in the chosen other bioactivity directions.

A further embodiment of the present invention is the ester scrambling method for the edible oil/phytosterol or edible oil/phytosterol/lecithin systems. In this reaction the heat treatment (with catalysis) allows the scrambling and exchange of ester groups between the triglicerides, the lecithins and the originally unesterified or esterified phytosterols. This corroborates the bioavailability and transport of the components involved.

A further embodiment of our present invention is the use of probiotics to obtain symbiotic compositions wherein to the compositions described earlier one or more probiotic strains of bacteria are added. This allows the formation of synbiotic compositions containing both prebiotic and probiotic elements. These compositions allow the selective food support of the already colonized intestinal bacteria as well as novel probiotics for colonizing mainly the large intestine (colon) and also supplying beneficial planktonic bacteria for the gastrointestinal system. The probiotic bacteria can be omitted or employed depending on the desired end-use of the compositions.

A further embodiment of our invention is the application of the method for the formation of compositions by spontaneous multicomponent supramolecular self-assembly for the preparation of cosmetics. The oils are employed individually or in a mixture of the following oils or butters: ostrich oil, evening primrose oil, jojoba oil, macadamia nut oil, shea butter, avocado oil, grapeseed oil, tamanu oil, rose hips oil, pomegranate oil, papaya seed oil, moring a oil, mango butter, argan oil, blackcurrant oil, almond oil, apricot kernel oil, borage oil, coconut oil, hazelnut oil, hemp seed oil, neem oil, olive oil, peach kernel oil, sesame oil, wheatgerm oil.

The compositions according to the invention are prepared in several forms that include beverages as well as solid medicaments, dietary supplements, food additives and foods, as well as cosmetics in various gel forms.

A further embodiment of this invention are cosmetic compositions with the usual skin-care and hair-care additives.

A further embodiment of present invention is pharmaceutical compositions. These pharmaceutical compositions can be prepared in the form of tablets, controlled release tablets, chewing tablets, enteric coated tablets, mucoadhesive vaginal tablets, capsules, gels, sols, solutions, tinctures, sprays, plasters, depending on the proposed application. In a typical embodiment of pharmaceutical compositions tablets are pressed. In these tablet form pharmaceutical preparations the active components are formulated together with diluents, excipients or carriers and disintegrants, selected from calcium carbonate, silicium dioxide, magnesium stearate, and fillers (lactose and dibasic calcium phosphate), and buffers (sodium bicarbonate, calcium carbonate, and sodium citrate), low substituted hydroxypropylcellulose, sodium carboxymethyl cellulose, microcrystalline cellulose, calcium carboxymethyl cellulose and croscarmellose sodium. Preferably, the composition also contains extra-granular components comprising silicon dioxide and a lubricant.

Mucoadhesive vaginal tablets can also be prepared from the basic compositions by directly compressing the natural chitosan, cross-linked with glutaraldehyde and if desired with sodium alginate, together with microcrystalline cellulose, sodium carboxymethylcellulose or the hydrophilic (hydroxypropyl methylcellulose [HPMC]).

Dietary supplements can also be prepared according to our invention comprising the prebiotic compositions with the usual nutritionally acceptable additives.

Further embodiment of our invention are prebiotic or synbiotic beverages that contain our prebiotic compositions together with natural fruit juices or other fluids, including dairy or non-dairy products with the usual nutritionally acceptable additives (sweeteners, acidulants, aromas, colorants).

A further embodiment of our invention are food items including prebiotic, probiotic or synbiotic hamburger, cheeseburger, pizza or other fast food. Due to the thermal stability of our compositions, any of these can be applied to the preparation of the hamburger buns and/or the hamburger meat. The probiotic component can be applied in the cheese or in the dressing. A typical burger according to our invention may contain 50% to 100% of the suggested daily dose of prebiotics, phytosterols, probiotics, certain vitamins and minerals. A prebiotic burger with a probiotic cheese or a probiotic dressing is a synbiotic food item (health food).

The following examples are given as illustrations only of the said invention and in no way should be construed as limiting the subject matter of the present invention.

### EXAMPLE 1

A phytosterol mixture (soy origin) (10 g) is heated with corn oil (20 g) for 2 hours at 100° C. Depending on the components a solid acid or other catalyst can be used. Then the mixture is cooled to 20° C. and added upon stirring into a mixture of lecithin (20 g, soy origin), water (20 ml) and L-lysine (10 g). Finally, this mixture is further mixed with 100 g prebiotic carbohydrates (80 g inulin, 10 g galacto-oligosaccharide, 8 g fructo-oligosaccharide and 2 g lactulose). Depending on the anticipated end-use, further components can be added that may include plant extracts and plant powders, vitamins, minerals, antioxidants and the usual fillers, stabilizers, adhesion modifiers.

### EXAMPLE 2

The method is followed described in Example 1 but corn germ oil is used.

### EXAMPLE 3

The method is followed described in Example 1 but corn fiber oil is used.

### EXAMPLE 4

The method is followed described in Example 1 but coconut oil is used.

### EXAMPLE 5

The method is followed described in Example 1 but pumpkinseed oil is used.

### EXAMPLE 6

The method is followed described in Example 1 but fish oil is used.

### EXAMPLE 7

The method is followed described in Example 1 but other edible oils or their mixtures are used.

### EXAMPLE 8

The method is followed described in Examples 1 to 7 but sunflower seed lecithin is used.

### EXAMPLE 9

The method is followed described in Examples 1 to 7 but egg lecithin is used.

### EXAMPLE 10

The method is followed described in Examples 1 to 9 but a tall oil phytosterol mixture is used.

### EXAMPLE 111

The method is followed described in examples 1 to 10 but the prebiotic mixture is 80 g of prebiotic carbohydrates (60 g inulin, 10 g beta-glucan, 8 g Aloe vera gel powder and 2 g tagatose).

### EXAMPLE 12

### Food Additive (Baking Mix)

To any of the basic compositions described in Examples 1-11, salt (NaCl) is added (50 g), followed by ascorbic acid (200 mg), a multivitamin mixture (1 g) and dry instant yeast (20 g) and the mixture thus obtained is thoroughly homogenized.

### EXAMPLE 13

### Probiotic Bakery Product (Bread)

To the baking flour or flour mixture (700 g) a baking mix, described in Example 12 is added (125 g), followed by water (0.3 to 0.4 liter) and the mixture thus obtained is kneaded into a dough. The amount of water depends on the flour or flour mix used. The dough is then processed and baked in an oven.

### EXAMPLE 14

### Synbiotic Product (Pastry)

In this product the dough is the prebiotic and the filling, applied after baking is the probiotic component. The dough is made by the use of any of the compositions described in Examples 1 to 11.

In a typical application, flour (380 g), composition according to Example 1 (120 g), dry yeast (20 g), sugar (25 g), lemon peel (grated, 30 g), eggs (2), margarine (50 g), milk (200 ml) is used to make a dough. This dough is levened, fried in 12 pieces in hot oil and filled (after cooling) with a cream containing the probiotic bacteria.

### EXAMPLE 15

### Dry Feed Additive

Any of the compositions described in Examples 1 to 11 is mixed with milled cereals (1 to 5 kg) and to this mixture oily seed industrial byproducts are added to obtain a mixture of 10 kg. This premix can be used in various fodder and dry feed mixtures.

### EXAMPLE 16

Any of the compositions described in Examples 1 to 11 is mixed with a proprietary composition of extracts of the following herbs and plants (20 g): Panax ginseng (red, Korean ginseng), Panax ginseng (white, Chinese ginseng), Rhodiola rosea (golden root), Panax quinquefolium (American ginseng), Eleutherococcus senticosus (Siberian ginseng), Cynara scolymus (artichoke), Uncaria tomentosa (Cat's claw), Lepidium meyenii (maca, Peruvian ginseng), Paullinia cupana (guarana), Croton lechleri (Sangre de Grado), Whitania somnifera (ashwagandha, Indian ginseng), Astragalus membranaceus (astragalus), Pfaffia paniculata (Brazilian ginseng, suma), Ginkgo biloba, Tabebuia impetiginosa (Pau d'arco), Echinacea purpurea, Peumus boldus (boldo), Gynostemma pentaphyllum (Jiaogulan, also known as Southern Ginseng or Xiancao), Sutherlandia frutescens (African ginseng), Aloe vera (aloe), Cistanche salsa, Cistanche deserticola, Codonopsis pilosula.

### EXAMPLE 17

The method is followed described in examples 1 to 11 but the oil employed is individually or in a mixture of the following oils or butters: ostrich oil, evening primrose oil, jojoba oil, macadamia nut oil, shea butter, avocado oil, grapeseed oil, tamanu oil, rose hips oil, pomegranate oil, papaya seed oil, moring a oil, mango butter, argan oil, blackcurrant oil, almond oil, apricot kernel oil, borage oil, coconut oil, hazelnut oil, hemp seed oil, neem oil, olive oil, peach kernel oil, sesame oil, wheatgerm oil. These compositions can be applied in the typical cosmetic bases in 1 to 90%.

### EXAMPLE 18

Pharmaceutical preparation containing one of the compositions of Examples 1 to 11, Example 16 and Example 17 in 20%, sodium carboxymethylcellulose 26%, sodium alginate 22%, microcrystalline cellulose 23%, hydroxypropyl methylcellulose [HPMC] 3% and chitosan 6%.

### EXAMPLE 19

Cosmetic composition containing one of the prebiotic compositions of Examples 1 to 11, Example 16 and Example 17 with the usual skin-care and hair-care additives. These compositions can be applied in the typical cosmetic bases usually in 1% to 90%. A typical application in moisturizing cream a composition described in Example 1 applied in the following manner: prebiotic composition 10 part, propylene glycol 4.0 part, methyl paraben 0.2 part, water 60.0 part, triethanolamine 2.0 part glyceryl sterate and PEG 6.0 part, stearate/stearic acid 6.0 part, cetyl alcohol 1.0 part, isopropyl myristate 15.0 part, propyl paraben 0.1 part, dimethicone 1.0 part, fragrance, coloring.

### EXAMPLE 20

Dietary supplement containing one of the prebiotic compositions of Examples 1 to 11, Example 16 and Example 17 with the usual nutritionally acceptable additives. In a typical embodiment of our invention 180 g of the prebiotic composition described in Example 1 was mixed with a proprietary mixture (100 g) of Cordyceps sinensis (Cordycaps), Lentinula edodes (Shitake), Ganoderma lucidium (Reishi), Grifola frondosa (maitake), Tremella fuciformis (Silver ear), Poria cocos (Hoelen), Hericium erinaceus (Lion's Mane), Agaricus blazei (Sun mushroom), Phellinus linteus (Mulberry yellow polypore), Tranetes versicolor, Coriolus versicolor (Turkey tails), Schizophyllum commune (Split gill), Inonotus obliquus (Cinder conk), oat bran, rice bran extracts and powders. After thoroughly mixing the composition thus obtained was filled in capsules or in bottles as loose powder.

### EXAMPLE 21

Beverage containing one of the prebiotic compositions of Examples 1 to 11, Example 16 and Example 17 with natural fruit juices or other fluids, including dairy or non-dairy products with the usual nutritionally acceptable additives. In a typical application the prebiotic composition described in Example 11 (160 g) was mixed with a proprietary mixture (5 g) of Pfaffia paniculata (Brazilian ginseng, suma), Ginkgo biloba, Tabebuia impetiginosa (Pau d'arco), Echinacea purpurea, Peumus boldus (boldo), Gynostemma pentaphyllum (Jiaogulan, also known as Southern Ginseng or Xiancao), Sutherlandia frutescens (African ginseng), Aloe vera (aloe), Cistanche salsa, Cistanche deserticola. The components were thoroughly mixed. A portion of this powder mixture (82.5 g) was added to 0.9 liter of orange juice with pulp. The final volume was corrected to 1 liter.

### EXAMPLE 22

### Prebiotic, Probiotic or Synbiotic Hamburger or Other Fast Food

Any of the compositions described in Examples 1 to 11 can be applied in the preparation of the hamburger bun and/or the hamburger meat. In the preferred embodiment of the invention one hamburger bun or one hamburger contains 8 g of the composition described in Example 1. The probiotic component can be applied in cheese or dressing.

A prebiotic burger with a probiotic dressing is a synbiotic food item (health food).

1. Synergistic compositions comprising prebiotic components selected from fructose polymers GFₙ and Fₘ, either containing a glucose (G) end-group, or without a glucose end-group, and one or more component of a group of prebiotics consisting of modified or unmodified starch and partial hydrolysates thereof, partially hydrolysed inulin, natural oligofructoses, fructo-oligosaccharides (FOS), lactulose, galactomannan and suitable partial hydrolysates thereof, indigestible polydextrose, acemannan, various gums, indigestible dextrin and partial hydrolysates thereof, trans-galacto-oligosaccharides (GOS), xylo-oligosaccharides (XOS), beta-glucan and partial hydrolysates thereof, together if desired with phytosterol/phytostanol components and their suitable esters, and if desired other plant extracts, mineral components, vitamins and additives.

2. Compositions according to claim 1 further comprising phytosterol/phytostanol components and their suitable esters.

3. Compositions according to claim 1 characterized in that the said further plant extracts or powders are one or more of those of Panax ginseng (red, Korean ginseng), Panax ginseng (white, Chinese ginseng), Rhodiola rosea (golden root), Panax quinquefolium (American ginseng), Eleutherococcus senticosus (Siberian ginseng), Cynara scolymus (artichoke), Uncaria tomentosa (Cat's claw), Lepidium meyenii (maca, Peruvian ginseng), Paullinia cupana (guarana), Croton lechleri (Sangre de Grado), Wliitania somnifera (ashwagandha, Indian ginseng), Panax japonicus (Japanese ginseng), Panax vietnamensis (Vietnamese ginseng), Panax trifolius, Panax pseudoginseng, Panax notoginseng, Malpighia glabra (acerola), Ylex paraguayiensis (Yerba mate), Astragalus membranaceus (astragalus), Stevia rebaudiana (stevia), Pfaffia paniculata (Brazilian ginseng, suma), Ginkgo biloba, Tabebuia impetiginosa (Pau d'arco), Echinacea purpurea, Peumus boldus (boldo), Gynostemma pentaphyllum (Jiaogulan, also known as Southern Ginseng or Xiancao), Sutherlandia frutescens (African ginseng), Aloe vera (aloe), Cistanche salsa, Cistanche deserticola (and other Cistanche sp.), Codonopsis pilosula (“poor man's ginseng.”), Nopal opuntia (Prickly pear cactus), Citrus sinensis (Citrus aurantium) and other members of the citrus family (lemon, lime, tangerine, grapefruit), Camelia sinensis (tea), Plantago psyllium (psyllium), Amaranth edulis and other amaranth sp. (amaranth), Commiphora mukul (guggul lipid), Serenoa repens, Serenoa serrulata (saw palmetto), Cordyceps sinensis (Cordycaps), Lentinula edodes (Shitake), Ganoderma lucidium (Reishi), Grifola frondosa (maitake), Tremella fuciformis (Silver ear), Poria cocos (Hoelen), Hericium erinaceus (Lion's Mane), Agaricus blazei (Sun mushroom), Phellinus linteus (Mulberry yellow polypore), Trametes versicolor, Coriolus versicolor (Turkey tails), Schizophyllum commune (Split gill), Inonotus obliquus (Cinder conk), oat bran, rice bran, linseed, garlic, Ceratonia siliqua (locust been gum or flour from the seeds of carob tree), Cyanopsis tetragonoloba (guar gum, EU Food additive code E412), Xanthomonas campestris (xanthan gum).

4. A composition according to claim 1 wherein the components are in a supramolecular arrangement.

5. A composition according to claim 4 characterized in that the micelle forming components of the supramolecular arrangement are a mixture of natural lecithins.

6. A composition according to claim 4 characterized in that the supramolecular arrangement is achieved in an oily phase.

7. A composition according to claim 6 characterized in that the oily phase is an edible oil or a mixture of edible oils preferably with omega-3-fatty acid content.

8. A composition according to claim 1 characterized in that one of the prebiotics is chicory inulin.

9. A composition according to claim 1 characterized in that the phytosterol components are of soy, corn or tall oil origin or a pure phytosterol, or a mixture of any of these phytosterols, phytostanols or esters thereof.

10. (canceled)

11. (canceled)

12. (canceled)

13. (canceled)

14. A method for the prevention, alleviation or treatment of diseases or disorders, said method comprising administering an effective amount of the composition of claim 1 to a patient.

15. The method of claim 14, characterized in that the said composition containing prebiotic components, phytosterols, lecithins and edible oils are applied for the change of lipid, and cholesterol and bile acid metabolism and transport, and blood lipid and cholesterol levels.

16. The method of claim 14, wherein the disease or disorder is selected from the group consisting of benign prostatic hyperplasia (BPH), unhealthy colonic and/or vaginal microflora, gastrointestinal damage caused by therapy with oral antibiotics or other oral antibacterial agents, allergies, and certain types of cancer.

17. (canceled)

18. (canceled)

19. The method of claim 14, wherein the prebiotic composition further comprises a probiotic thus providing a symbiotic preparation.

20. (canceled)

21. (canceled)

22. (canceled)

23. The method of claim 14 for enhancing an immune response which comprises administering the composition in an amount sufficient to enhance a detectable immune response.

24. (canceled)

25. The method of claim 14, further comprising administering cholesterol lowering drugs.

26. The method of claim 14, further comprising administering antibiotic or (synthetic) antibacterial therapy.

27. (canceled)

28. Methods for the preparation of compositions and products described in Examples 1 to 22.

29. The method of claim 14 wherein the composition is in the form of a food product, beverage product, nutritional or fodder additive preparation, pharmaceutical preparation, cosmetic, infant formula, or immune modulant.
